(19) United States
(12) Patent Application Publication
(10) Pub. No.: US 2022/0080060 A1
(43) Pub. Date: Mar. 17, 2022

(54) DISINFECTION SYSTEM AND METHOD

(71) Applicant: AI Data Innovation Corporation, Bellevue, WA (US)

(72) Inventors: Patrick McKinley JARVIS, Redmond, WA (US); Ke WANG, Charlottesville, VA (US)

(21) Appl. No.: 17/473,332

(22) Filed: Sep. 13, 2021

Related U.S. Application Data
(60) Provisional application No. 63/077,466, filed on Sep. 11, 2020.
(60) Provisional application No. 63/142,358, filed on Jan. 27, 2021.

(51) Int. Cl. A61L 2/00; A61L 2/28

(52) U.S. Cl. CPC A61L 2/0047; A61L 2/0076; A61L 2202/14; A61L 2202/11; A61L 2202/20; A61L 2/28

(57) ABSTRACT

A disinfection monitoring system and method for performing monitoring of a disinfection system is provided. The disinfection monitoring system may include one or more image capture devices configured to capture a set of images or videos of an object to be disinfected, such as a user's hands, fingers, and body during an disinfection procedure. An AI module performs detection and detection-based tracking from the captured image or video to determine a quality of the disinfection procedure. The detection and detection-based tracking is performed using keypoint or key area based detection of the user's hands, fingers, and body during the disinfection procedure. A disinfection data collection system generates a ground-truth score from the set of images or videos that indicate a quality of the overall disinfection procedure and/or a quality of each step in the overall disinfection procedure. The ground-truth score is used to train the disinfection monitoring system.

### TECHNICAL FIELD

The disclosure relates to disinfection systems and method, in particular systems and method for human disinfection including handwashing applications.

### BACKGROUND

The outbreak of the severe acute respiratory syndrome coronavirus 2 (SARS-COV-2) in 2019, and the ensuing epidemic of 2020, has increased and highlighted the importance of and need for improved disinfection systems and methods. There is a strong consensus that the importance of handwashing in preventing and mitigating infection cannot be overemphasized, as the novel coronavirus can survive on a variety of surfaces for several days, such as doorknobs, tables, desks, etc., and can be transmitted after a person unknowingly touches a contaminated surface and then touches their nose, eyes, or mouth. The novel coronavirus can also be transmitted from accumulations of the virus on the outside of a person's mask, such as when the user removes or adjusts the mask with their hands and then touches their face or a surface where contact may occur.

Handwashing is further important after numerous events, such as nose blowing, coughing, sneezing, before, during, and after caring for a sick person, after being in a public area, before, during, and after food preparation, after changing diapers or cleaning up or playing with children, after touching or feeding an animal, after handling products on a shelf in a store, or after cleaning or taking out garbage, to name a few. Handwashing before, during, and after such events helps to prevent spread of the coronavirus from a sick person, especially an asymptomatic person who may be unaware of their ability to spread the coronavirus or other infectious diseases, to others.

The importance of disinfection procedures such as handwashing is not limited to the novel coronavirus, but rather is an imperative measure in healthcare, food service, retail shopping, laboratory science, and other settings, and is an effective measure against influenza and other major disease outbreaks.

The World Health Organization (WHO) has proposed a 12-step process that is accepted as the standard for thorough handwashing, including the steps of 1) wet hands with water, 2) apply enough soap to cover all hand surfaces, 3) rub hands palm to palm, 4) right palm over left dorsum with interlaced fingers and vice versa, 5) palm to palm with fingers interlaced, 6) backs of fingers to opposing palms with fingers interlocked, 7) rotational rubbing of left thumb clasped in right palm and vice versa, 8) rotational rubbing, backwards and forwards with clasped fingers of right hand in left palm and vice versa, 9) rinse hands with water, 10) dry thoroughly with a single use towel, 11) use towel to turn off faucet, 12) and your hands are safe, performed for the length of time it takes to sing “Happy Birthday” twice. The soap disrupts the lipid envelope around the coronavirus, rendering it unable to infect a person, and also facilitates the removal of the coronavirus from the washed surface. Other health organizations have proposed varying multi-step procedures.

Problematically, handwashing techniques, duration, and quality vary widely among people and at different times. Most people who practice handwashing diligently may be completely unaware that their handwashing technique is inadequate, for example. Missing or poorly implementing one or more steps will lead to a poor performance of the whole washing procedure. In other words, there is a need for a system that can adequately monitor one or more steps, and in embodiments every step of a disinfection procedure, to prevent a single misstep from leading to inadequate disinfection. But in the absence of robust means for monitoring the quality of handwashing, the problem of poor-quality handwashing plagues numerous individuals and industries, especially industries where handwashing quality is imperative to prevent the spread of disease, such as healthcare and food service.

It has been found from observational studies that only about 5% of people properly wash their hands, and a substantial number of people do not wash their hands at all after using the bathroom (10%) and a substantial number of people do not use soap when washing (25%). On average, people wash for only six seconds rather than the recommended 20. However, the known deficiencies of the average person's handwashing quality notwithstanding, given the sensitivity of monitoring people's behavior at bathroom sinks, there is no simple or obvious solution for instilling good handwashing techniques in people at a local site or society-wide.

Likewise, disinfection procedures such as disinfecting objects or surfaces such as table surfaces, shopping carts, shelves and displays in a retail setting, medical devices and beds, and other surfaces, are highly subject to varying levels of quality and compliance by various users. There is no existing method for automatically quantifying the quality of a disinfection procedure for such surfaces.

Previous efforts to monitor handwashing have focused variously on providing cameras for assessing the movement of hands using hand-tracking algorithms or providing a UV-visible material that can be scanned to determine the cleanliness of a user's hands. It is straightforward enough to capture videos of a handwashing procedure, however, existing modalities for handwashing-quality tracking have thus far failed to address the basic issues relating to privacy, efficacy, and scalability, such as the ability to remove the user's face or other sensitive or irrelevant information while tracking the user's hands in real-time. Existing modalities fail to provide a privacy-ensuring handwashing-monitoring approach that accurately monitors a user's hands to ensure that the requisite gestures have been followed for the requisite amount of time.

Existing approaches that attempt to provide an artificial intelligence solution to the problem of monitoring handwashing or other disinfection procedures also fail to effectively and automatically sanitize and remove sensitive or irrelevant components of captured images, critically limiting the ability of any organization to handle privacy concerns while ensuring proper disinfection. For this reason, certain existing approaches consist solely in providing a handwashing training console or station where users can practice handwashing at a controlled console, but there is no known or attempted way to apply such a console to existing disinfection facilities such as wash stations and restrooms in healthcare facilities and food preparation facilities.

Another problem with existing solutions is the absence of agreement regarding optimum practice for disinfection practices such as handwashing. There is no consensus regarding how much time each step of the WHO-recommended 12-step process should take. In the absence of a quantitative method for standardizing disinfection activities like handwashing, disagreement abounds regarding the best and most effective method of handwashing, and many people are unaware of how to best protect themselves and others.

Yet another problem with existing solutions is the inherent difficulty of capturing and properly detecting a pose of a user's hands in a handwashing or other disinfection process from a two-dimensional video feed. Existing attempts to assess the quality of a handwashing procedure, for example, have poor reliability because of misdetection events caused by occlusion of the hands from a camera, for example by one hand which is being used to wash the other hand, by soap and/or water on one of both of the hands, by a plumbing structure such as a faucet, or otherwise. Frames of a video feed that are so occluded often result in misdetection and reduced granularity of an analysis, as fewer frames are available to properly assess a procedure quality.

Manual efforts to provide such assessment of captured videos of disinfecting procedures are necessarily labor-intensive and not feasible for real-time analysis. Manual assessment and editing further necessarily compromise the privacy of individual users as the videos are being edited to remove personal or sensitive information.

In view of the above, there is a need for an improved system and method for automatically and quantitatively assessing the quality of a disinfecting procedure, such as handwashing, while ensuring the privacy and sensitivity of users involved.

### SUMMARY

Disinfection monitoring systems and methods of the present disclosure advantageously address the known problems of existing handwashing systems and methods. The disinfection system and method embodiments are also applicable to, e.g., indoor disinfection (such as of furniture, toys, appliances, countertops, toilets, sinks, foodstuffs, medical equipment, or clothing, for example), outdoor disinfection (such as of playground equipment, packages, or deliveries, for example), or human body disinfection (such as of arms, legs, feed, or faces of school children, cashiers, food preparation workers, health professionals, or otherwise). While reference is made specifically to handwashing throughout the disclosure, it will be understood that any suitable activity is contemplated.

The disinfection monitoring system and method embodiments may advantageously provide real-time feedback of compliance with one or more disinfection standards or protocols, such as providing real-time feedback of whether a user has properly completed each of the 12 recommended handwashing steps taught by the WHO. The system may be configured to provide an alert to the user indicating proper compliance (and that the user may return to or proceed with a particular work task), or an alert that the handwashing has not been properly completed. In embodiments, a particular step that was failed and needs attention can be indicated. In other embodiments, the system may provide a record of compliance for a particular user.

The system may track the identity of the user to provide a compliance record. In embodiments, an employer (such as a hospital, restaurant, or retailer) may track employees' compliance with handwashing requirements for purposes of regulatory compliance, security, marketing, or any other suitable purpose. The system may comprise any suitable modality for identifying the user, such as requiring a biometric identification credential, such as a smart card, retinal scan, fingerprint scan, facial recognition scan, or otherwise at the beginning of the handwashing procedure. The processor may receive the biometric identification credential and compile the same in a central database.

The embodiments may comprise at least one image capture device and at least one processor configured to evaluate at least one image obtained from the at least one image capture device. The at least one processor may advantageously obscure, remove, or otherwise sanitize a captured image such that sensitive or personal details are protected and/or excluded, without compromising the ability of the system to accurately monitor the quality of a handwashing incident. This may be accomplished substantially in real-time to provide on-the-spot feedback to the user, particularly to require the user to repeat a step if necessary. In embodiments, the analysis may be conducted offline, in the cloud, remotely, or after the fact, as suitable.

In embodiments, the image capture device of the disinfection system may comprise a motion-trigger system or a sensor based system (such as a pressure sensor on a sanitizer bottle) to activate the system when a user begins the disinfection process. The processor may be configured to, upon or after capturing an image or video of an activity within the field of view of the image capture device, to determine whether the activity is an activity of interest, for example handwashing as opposed to brushing teeth, shaving, applying, or removing makeup, or otherwise. The determination may be made prior to any further assessment as described herein. Upon a determination that the activity monitored is not an activity of interest, the captured image or video may be deleted. Upon an alternative determination that the activity monitored is an activity of interest, the image or video may be saved or may be subjected to further processing, as described herein.

In alternative embodiments, prior to capturing an image or video of the process, the processor may assess a captured preliminary image or video to assess whether to continue with capturing the image or video. For example, the processor may determine from the preliminary image whether the activity being conducted is the intended activity, such as handwashing. In embodiments relating to handwashing, the processor may assess a captured preliminary video and assess that the person is brushing their teeth, and deactivate the image capture device. The image capture device may be configured to capture a second preliminary image or video upon a predetermined time interval to re-assess whether the user has commenced the activity of interest, such as if a user begins handwashing after they have finished shaving or brushing their teeth. Upon a determination that the preliminary image or video is an activity of interest, the image capture device can be activated to capture a primary image or video of the activity of interest.

The processor may be configured to analyze the captured video frame-by-frame to remove entire frames that are not relevant to the activity of interest. For example, the processor may be configured to remove frames before the handwashing steps have been commenced, frames between steps, or frames after the handwashing has been completed.

Additionally, or alternatively, the processor may be configured to identify and blur or remove static background. In embodiments relating to handwashing, the image capture device may be oriented substantially downwardly such that the user's hands and arms and the underlying sink are primarily captured, or substantially horizontally such that the user's face, body, and bathroom features are captured, or any other orientation. The processor can advantageously be configured as described herein to identify static features between the frames of the captured video and to blur or alternatively remove such features from a final video product. The final video product can thereby focus solely on the relevant features, i.e., the hands that are being washed, while blurring or removing the surroundings, which ensures privacy. The video may be stored on the database as a blurred or removed version of the original video, thereby preventing that any versions of the video that show static features are stored on the system.

The processor may also or alternatively be configured to identify and blur or remove human faces. The image capture device may be oriented so as to capture images that include not only a person's hands but also the person's face, and/or the face of persons passing by in the same washing facility, e.g., a public restroom. The processor can advantageously be configured as described herein to identify one or more human faces in one or more of the frames of the captured video and to blur or alternatively remove altogether the one or more faces, thereby ensuring privacy for the user. The video may be stored on the database as a blurred or removed version of the original video, thereby preventing that any versions of the video that show one or more human faces are stored on the system. In addition, any other human identifying features such as birthmarks, tattoos, or other user identifying parts can also be blurred or removed as discussed in relation to the user's face.

In embodiments, two distinct image capture devices with different positions and/or poses may be used: a first camera configured to capture an image that includes a user's face for identification using biometric information such as facial recognition information or a retinal scan, and a second camera configured to capture an image that includes a user's hands and arms but not their face or individuals walking behind the user. The images from the first camera may be used solely for identification purposes and not stored on the system, while the images from the second camera may be used for analysis of handwashing procedure without including sensitive information or background. Images obtained from the first and/or second camera may be processed as described above to remove static background and/or sensitive information such as a user's face.

While first and second cameras oriented and configured to capture different positions, poses, or parts of a user have been described above, it will be appreciated that any number and arrangement of image capture devices may be utilized as suitable. For example, a single camera may be used to capture both a user's face for biometric authentication and to capture the user's hands and/or arms, with the captured images or frames of a video feed processed locally, remotely, or in any suitable location. In other embodiments, a third and/or fourth camera may be oriented to capture the hands and/or arms from a different angle than the second camera and/or the first camera so as to reduce the incidence of occlusion of hand, finger, and/or human keypoints or key areas from captured images.

In embodiments, the third and/or fourth camera may be oriented in a substantially transverse orientation relative to the second camera so as to capture an image or images of the user's hands and/or arms from a different angle. Any suitable offset or orientation of the cameras relative to each other may be utilized. For example, if the second camera is oriented substantially downwardly, the third camera may be oriented horizontally and/or from a side of the sink. In embodiments, two cameras may be oriented generally downwardly but may be offset from each other by, for example, 45°. Additionally, the cameras may have different scopes, i.e., differently sized fields of view. One camera may be configured with a wide angle or field of view so as to capture an entirety of a user's body for human keypoint or key area detection, whereas another camera may be configured with a smaller angle or field of view so as to capture only the hands, fingers, and/or arms, for example. The one or more cameras may be RGB 2D cameras, IR cameras or Depth cameras, or any other suitable camera.

The one or more cameras may be configured to capture different types of images, For example, the cameras may be configured one or more of an RGB (i.e. red, green, blue) or truecolor image, a cyan, magenta, yellow (“CMY”) image, a cyan, magenta, yellow, key/black (“CMYK”) image, a hue, saturation, and intensity (“HSI”) image, an XYZ image, a UVW image, YUV, YIQ, or YCbCr image, a YDbDr image, a DSH, HSV, HLS, or HIS image, a Munsel color space image, a CIELuv image, a CIELab image, a SMPTE-C RGB image, a YES (Xerox) image, a grayscale image, a digital infrared image, or any other suitable type of image.

In embodiments, the captured images may be processed locally to remove any identifying or sensitive information before transmitting the captured images to a remote or cloud processing or storage location. That is, the disinfection system may comprise locally a processor and/or storage medium with instructions for removing or obscuring background, facial features, identifying features, and/or other sensitive details from captured images before the captured images are ever transmitted to a remote or cloud location. In embodiments, the system may perform keypoint or key area detection upon one or more captured image locally and transmit only detected bounding boxes and/or keypoints or key areas in lieu of image data. The system may be configured to perform handwashing analysis of the procedure using the keypoint or key area detection results, i.e., the bounding boxes and/or keypoints or key areas, remotely.

This allows the disinfection system to ensure privacy, security, and compliance with privacy-related statutes. In particular embodiments, the system may perform a monitoring procedure of a disinfection procedure and provide feedback to a user without storing any images of the user on the system, thus ensuring improved privacy and security.

The system and processor may be configured in embodiments with an algorithm to classify each frame of a captured video as a particular step of a standard disinfection procedure and to score the completeness of the classified step. The standard disinfection procedure can be in embodiments the aforementioned 12-step handwashing procedure specified by the WHO or any other suitable procedure. The system may include an interface, including for example a screen and/or speaker, that may communicate to a user how closely their disinfection practices satisfied the standard disinfection procedure and in particular which step, if any, was problematic. In embodiments the interface may comprise a screen of a smartphone or other device from which the user may access a mobile application to receive feedback. In embodiments, the mobile application may be configured to provide a notification to a user in the form of a sound, a vibration, and/or an indicium on the screen to indicate whether the user has passed the assessment, which step(s) of the procedure may require improvement and/or a completeness of the step(s), and/or any other suitable indicium.

The system may further log the result of the activity of interest for future analysis, compliance reporting, and trend assessment. For example, a particular user's scores may be assessed over time to determine whether the user is progressing toward reliable compliance. Other data such as the time of day, day of the week, or other factors may be logged as well.

The algorithm may perform detection-based tracking of a user's fingers based on a motion recognition of finger motion patterns, with a step-by-step analysis of strengths patterns, which may include a combination of 1) duration of the step and 2) amplitude. The algorithm may leverage a machine learning model, such as but not limited to a data-driven deep learning model, for finger detection using object tracking or keypoint tracking techniques that considers clutter and misdetection for individual hands/fingers and trained using automated feature selection.

The detection-based tracking of a user's fingers, arms, or other body parts may be performed using keypoint or key area-based tracking and/or object tracking. For example, the detection-based tracking may utilize a machine learning model, such as but not limited to a deep neural net model, to obtain one or more frames of a video feed, overlay one or more keypoints or key areas and/or bounding boxes to the frame to identify a user's arms, wrists, hands, and/or fingers or any other suitable type, number, and combination of keypoints or key areas, and to determine a pose, such as one of the steps of the WHO-recommended handwashing procedure, in a particular frame. The keypoints or key areas may be predefined so as to correspond to a desired feature of a user, such as predetermined keypoints or key areas including joints such as the shoulder, elbow, wrist, and/or knuckles, fingertips, and/or other keypoints or key areas as suitable. In embodiments, keypoints or key areas of the user's head, face, torso, legs, and/or feet may also be used.

A number of successive frames of a video feed automatically identified by the system as pertaining to a particular pose may be determined such that a duration of each pose of a plurality of poses of a predetermined procedure may be determined and assessed against the procedure. For example, in embodiments the system may require a minimum of three, five, or any number of seconds on each pose of a predetermined procedure. The system may require a different duration for different poses as suitable.

In embodiments, hand detection is combined with human keypoint or key area detection to provide improved reliability of detection. By combining human keypoint or key area detection with hand detection, the hand detection analysis may be anchored by the human keypoint or key area detection analysis. For example, in one or more frames of a video feed, one or both of a user's hands may be occluded (for example, by the other hand), by soap and/or water, or by a structure such as a faucet of a sink. In such instances the arms, including the elbow and/or wrists, may still be visible. By performing human keypoint or key area detection of the user's arms, for example, in parallel with hand detection, the system of embodiments may estimate a pose and/or a step of a handwashing procedure, including amplitude and duration, even when one or both hands are partially or wholly occluded in one or more frames by estimating a pose on the basis of the location and movement patterns of human keypoints or key areas, including the elbow and/or wrist. This may result in a greater quantity of discrete frames of a video feed being available for analysis of the user's handwashing technique.

Hand detection and/or human keypoint or key area detection may be performed using one or more pretrained models, such as machine learning models, in embodiments one or more deep neural net models. In embodiments, hand and finger detection is performed by a distinct model compared to a model for human keypoint or key area detection. For example, a human keypoint or key area detection model may be configured to detect and identify predefined keypoints or key areas on each presenter. There may be any suitable number of keypoints or key areas, for instance 17, 25, or any other suitable number. The keypoints or key areas may be predefined to correspond to a desired feature of a person, such as joints including the hip, knee, ankle, wrist, elbow, and/or shoulder, body parts such as the foot tip, hand tip, head top, chin, mouth, eyes, and/or ears, or any other suitable feature. Any suitable combination or number of features may be utilized.

By contrast, in embodiments, a model for hand detection may utilize a pretrained model for hand-related keypoint or key area detection involving one or more keypoints or key areas pertaining to the hand, such as but not limited to 1) a wrist keypoint or key area, 2) a scaphoid keypoint or key area, 3) a trapezium keypoint or key area, 4) a first metacarpal keypoint or key area, 5) a first proximal phalange keypoint or key area, 6) a thumb tip keypoint or key area, 7) a second metacarpal keypoint or key area, 8) a second proximal phalange keypoint or key area, 9) a second middle phalange keypoint or key area, 10) an index finger tip keypoint or key area, 11) a third metacarpal keypoint or key area, 12) a third proximal phalange keypoint or key area, 13) a third middle phalange keypoint or key area, 14) a middle finger tip keypoint or key area, 15) a fourth metacarpal keypoint or key area, 16) a fourth proximal phalange keypoint or key area, 17) a fourth middle phalange keypoint or key area, 18) a ring finger tip keypoint or key area, 19) a fifth metacarpal keypoint or key area, 20) a fifth proximal phalange keypoint or key area, 21) a fifth middle phalange keypoint or key area, and 22) a pinkie finger tip keypoint or key area.

In embodiments where human keypoint or key area detection is combined with hand and finger detection, one or more keypoints or key areas pertaining to a user's body, including arm-related keypoints such as elbow, wrist, and hand tip may be detected in parallel with one or more keypoints or key areas pertaining to a user's hands, such as an elbow and a wrist keypoint or key area detected and analyzed in combination with all or some of the 22 hand-related keypoints or key areas noted above.

In any of the above-described implementations, a detected keypoint corresponding to a predetermined feature of a user's body may be represented or detected as a key area, e.g., a circular area, surrounding a likely keypoint with a probability confidence interval, such as one sigma—corresponding to one standard deviation. The key areas may indicate a probability that each pixel in the input image belongs to a particular keypoint. The use of key areas may be advantageous in embodiments as relying on detected key areas allows the system and method to skip a step of generating a mask value and rather includes all or substantially all pixels of a key area in the determination of a pose amplitude and/or duration as described herein.

In embodiments, the system may need a large amount of video data to be trained to achieve a good performance. Therefore, in such embodiments a data collection method or procedure is established to collect the data properly. An automatic recording system described in and [0023] may be used in the in a data collection procedure. The personal identifying information (PII) removal method described in [0025] and [0026] may also be used in the data collection procedure. In the embodiments, a non-limiting goal of the data collection method or procedure may be to obtain a number of videos of the disinfection procedure as captured by the one or more cameras. Each of the videos may receive a corresponding ground-truth score, which may be generated by a human evaluation or by the system. The ground-truth score indicates the quality of the overall procedure and/or quality of each step in the disinfection process and may be used as training data by the AI modules of the system.

In embodiments, the ground-truth score of the disinfection quality may be obtained using a UV light system. The UV light system may be configured with a bottle of UV-active material, one or more UV-emitting light sources, and a second image capture device that captures images before and after a disinfection event such as handwashing. The UV light system may be configured to capture an image prior to handwashing and after handwashing and output a percentage of cleaned area, as determined based on the amount of UV-active material removed during handwashing, as the score of the handwashing procedure. The ground-true disinfection quality may be obtained through other methods such as expert scoring.

### BRIEF DESCRIPTION OF THE DRAWINGS

These and other features, aspects, and advantages of the present disclosure will become better understood regarding the following description, appended claims, and accompanying drawings.

FIG. 1A is a diagram of a disinfection monitoring system according to an embodiment of the disclosure.

FIG. 1B is a diagram of a disinfection data collection system according to an embodiment of the disclosure.

FIG. 2 is a diagram of a disinfection monitoring method according to an embodiment of the disclosure.

FIG. 3A is an annotated image generated by a disinfection system according to an embodiment of the disclosure.

FIG. 3B is an annotated image generated by a disinfection system according to the embodiment of FIG. 3A.

FIG. 3C is an annotated image generated by a disinfection system according to the embodiment of FIG. 3A.

FIG. 3D is an annotated image generated by a disinfection system according to the embodiment of FIG. 3A.

FIG. 3E is an annotated image generated by a disinfection system according to the embodiment of FIG. 3A.

FIG. 4 is a perspective view of a disinfection system according to an embodiment of the disclosure.

FIG. 5 is a diagram of a disinfection data collection method according to an embodiment of the disclosure.

### DETAILED DESCRIPTION OF VARIOUS EMBODIMENTS

### A. Overview

A better understanding of different embodiments of the disclosure may be had from the following description read with the accompanying drawings in which like reference characters refer to like elements.

While the disclosure is susceptible to various modifications and alternative constructions, certain illustrative embodiments are in the drawings and are described below. It should be understood, however, there is no intention to limit the disclosure to the specific embodiments disclosed, but on the contrary, the intention covers all modifications, alternative constructions, combinations, and equivalents falling within the spirit and scope of the disclosure.

It will be understood that unless a term is expressly defined in this application to possess a described meaning, there is no intent to limit the meaning of such term, either expressly or indirectly, beyond its plain or ordinary meaning.

### B. Various Embodiments and Components for Use Therewith

Disinfection system and method embodiments are described herein. A disinfection system and method according to the disclosed embodiments advantageously provides an accurate and private method for ensuring compliance with a hygienic practice, such as handwashing. The system and method can operate in substantially real-time or provide after-the-fact analysis of a hygienic practice, and the system may assess the hygienic practice by processing one or more captured images locally or remotely.

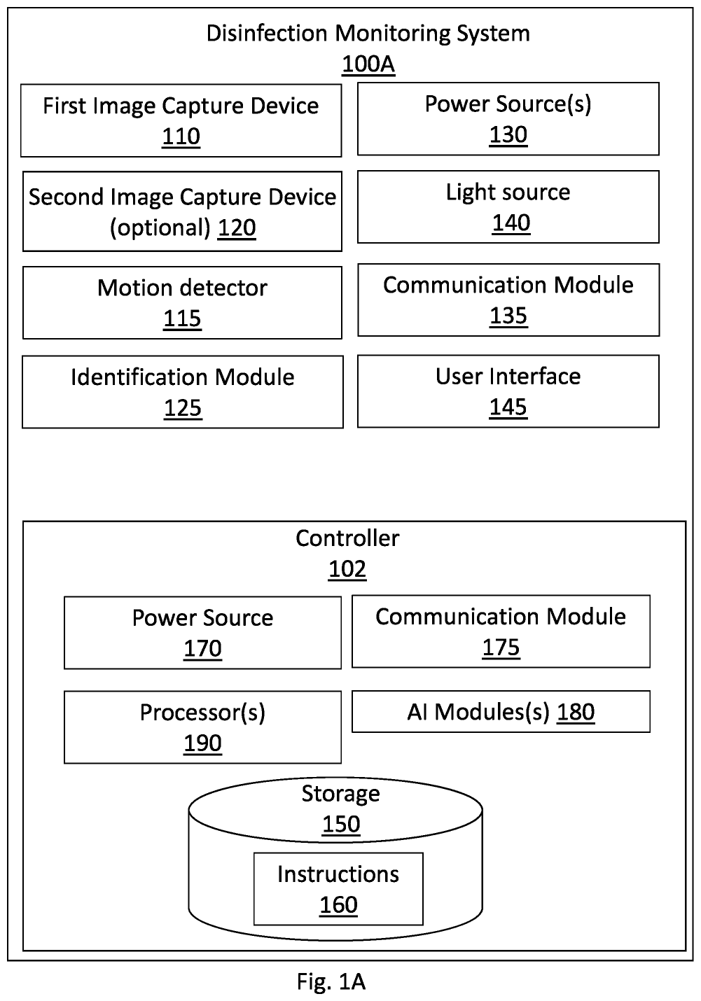

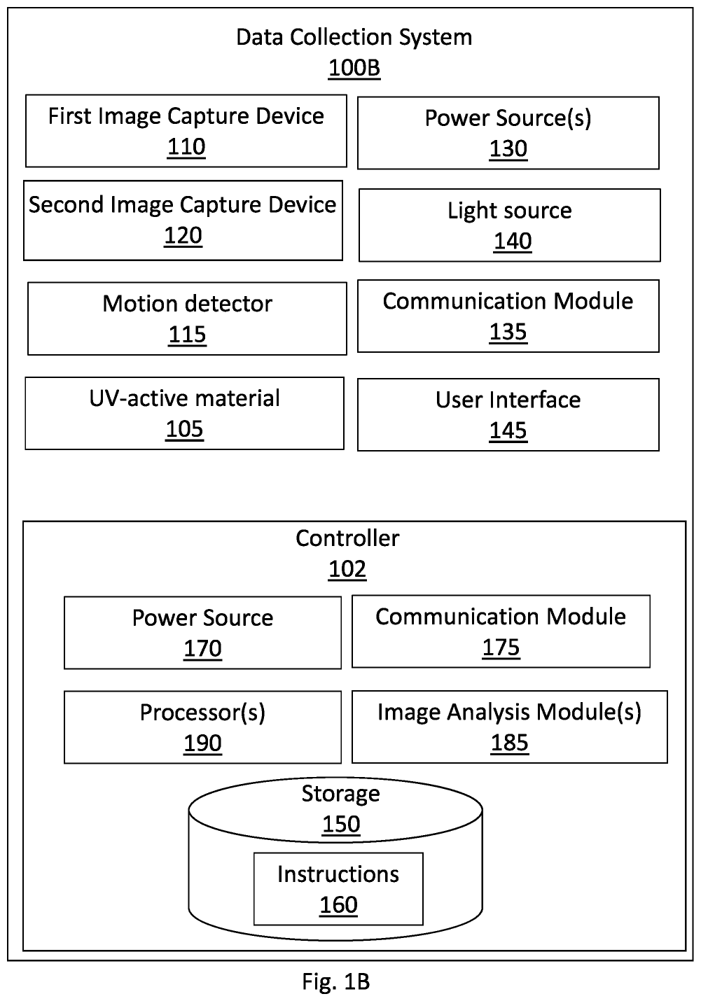

FIG. 1A is a diagram of a disinfection monitoring system 100A according to an embodiment of the present disclosure that may be used in particular to monitor a disinfection process or method in real-time. The disinfection monitoring system 100A may include a first image capture device 110. The first image capture device 110 may be any suitable image capture device, such as a digital camera configured for capturing a single image or a video comprising a plurality of frames. The first image capture device 110 may be configured with an attachment component so as to be mountable in any suitable position. The attachment component may be any suitable component, such as hardware components including a wall or ceiling mount that can attach using one or more screws or other components.

The attachment component may comprise one or more lockable joints cooperating with one or more body segments that allow the camera to be pivoted or swiveled to a desired position. For example, the one or more lockable joints can be unlocked to pivot the attachment component such that the camera 110 points toward towards a sink, and then locked to prevent unwanted movement of the camera 110 relative to the sink. The attachment component may be configured to allow the camera 110 to be readily installed in any existing facility, such as an existing restroom of a healthcare facility or restaurant, and positioned as suitable for said facility. In embodiments, the camera 110 may be positioned to face downwardly towards the sink only, while in embodiments it may be more suitable to position the camera 110 to face substantially horizontally toward the person using the system 100A. More than one camera 110 may be provided as suitable, such as to capture the person performing a disinfection activity from different angles. In some embodiments, the disinfection system 100A may include a second image capture device 120, although this is not required. The second image capture device 120 may be any suitable image capture device and may also include an attachment component as discussed in relation to image capture device 110. The image capture devices 110, 120 may be RGB 2D cameras, IR cameras or Depth cameras, or any other suitable camera.

In embodiments the first and/or second image capture devices 110, 120 may be configured to cooperate with a light source 140, which may or may not be integrated with the first and/or second image capture devices 110, 120. For example, the light source 140 may be a light emitting diode (LED) light configured to provide a flash for the first and/or second cameras 110, 120, or any other suitable light source.

An identification module 125 may cooperate with the first and second image capture devices 110, 120, and may receive at least one identification credential from a user for the purpose of identifying the user to the system 100A. The identification module 125 may comprise any suitable modality for receiving identification credentials such as a smart card, a passcode or password, a retina scan, iris scan, a fingerprint scan, a voice recognition scan, facial recognition modalities, or any other suitable identification credential. The identification module 125 may comprise an image capture device, a card reader, a fingerprint scanner, a retina scanner, a microphone configured for voice recognition, and/or any other suitable modality. The identification module 125 may be configured to receive a combination of identification credentials, such as both a fingerprint scan and a passcode, or a smartcard and a voice recognition credential. Any number and combination of any suitable credentials may be utilized.

The first and second image capture devices 110, 120, the motion detector 115, the identification module 125, and a light source 140 may be operatively connected to a controller 102 by a communication module 135 which may be any suitable connection modality, including a wired connection or a wireless connection. The communication module 135 may deliver the captured images from the first and second image capture devices 110, 120, one or more received identification credentials from the identification module 125, and/or a motion detection signal from the motion detector 115 to the controller 102, and may deliver a control signal from the controller 102 to the first and second image capture devices 110, 120 and/or to the light source 140. For example, the communication module 135 may facilitate a transmission of a control signal from the controller 102 to the light source 140 to activate the light source in response to a determination by the controller 102 that a motion detection signal from the motion detector 115 that a person is present at the sink and an image or video should be captured. The communication module 135 may also be connected to a cloud server to process or store the data.

The controller 102 may be local to the disinfection events, such as installed with the image capture devices 110, 120, and the light source 140 in situ, such as in a restroom or proximate a sink. In alternative embodiments, the controller 102 may be remote to the disinfection events, such as at a central location external to the restroom or sink. For example, the controller 102 may be located at a server corresponding to a facility where multiple disinfection events take place, such as a central server located in a hospital with many sinks and restrooms. In embodiments, a single controller 102 corresponds to a single set of image capture devices 110, 120 at a single restroom facility or sink, while in other embodiments a single controller 102 corresponds to a plurality of image capture devices 110, 120 located at different sinks in a single restroom or a single building.

A power source 130 may be provided for the first and/or second image capture devices 110, 120, the motion detector 115, the identification module 125, the light source 140, the communication module 135, and/or the user interface 145, and may comprise a battery power source or a wired connection to an existing power source, such as a power outlet in a restroom facility.

The user interface 145 may comprise any suitable user interface for communicating with a user. In embodiments, the user interface 145 may comprise an electronic display such as an electroluminescent (ELD) display, a liquid crystal display (LCD), a light emitting diode (LED) display, a plasma display, a quantum dot display, a touch screen such as a resistive touch screen, a surface capacitive touch screen, a projected capacitive touch screen, a surface acoustic wave (SAW) touch screen, an infrared (IR) touch screen, or any other suitable electronic display. The user interface may comprise one or more user input options or modalities, such as buttons or a keyboard, that allow a user to input information such as identification information.

In embodiments where the user interface 145 comprises a type of display as discussed previously, the display may be configured to include or display various animations or illustrations of each step of a disinfection process, such as the aforementioned 12-step handwashing procedure specified by the WHO. The animations or illustrations may thus guide a user on how to properly perform each step of the disinfection process.

The user interface 145 may be configured to display a signal indicating whether the user has successfully complied with a requirement for the disinfection procedure. In instances when a user has not successfully complied with the disinfection procedure, particularly regarding a particular step, the user interface 145 may display a number or image corresponding to a step of the disinfection procedure and instructions for improvement. The display may include an animation or illustration of the step. The user interface 145 may receive one or more control signals from the controller 102 through the communication module 135 for indicating to the user whether the disinfection procedure has been successfully completed and/or which step(s) of the procedure require attention should the user need to repeat the disinfection procedure. In embodiments, the system 100A may require a user to repeat an entirety of a disinfection procedure upon determining that one or more steps have been failed, or may require the user to repeat only the failed one or more steps.

In embodiments, the system 100A may be configured to provide feedback after each particular step rather than waiting until the entire procedure has been completed to provide feedback. For example, if the system 100A detects that the user has conducted a particular step such as a recommended step of rotational rubbing of clasped fingers against the opposite palm, either using poor technique or for an insufficient duration, the system 100A may prompt the user to repeat said step until it has been satisfactorily completed before guiding the user to other steps.

The controller 102 may advantageously be located local and proximate to the image capture devices 110, 120, the identification module 125, the motion detector 115, the light source 140, and the user interface 145, or may be located remote. The controller 102 may comprise a power source 170 which may be a battery-based power source or a power source that draws power from an existing power supply, such as through a power outlet in the pertinent facility where the system 100A and controller 102 are located. The power source 170 may operate the controller 102 and components thereof to perform the functions described herein.

The controller 102 may comprise a communication module 175 configured to receive information and signals from the first and second image capture devices 110, 120, motion detector 115, identification module 125, and user interface 145 through the communication module 135, and/or to provide one or more control signals from the controller 102 to the communication module 135 for activating and controlling the image capture devices, motion detector, identification module, light source, and user interface 110, 120, 115, 125, 145. This may be accomplished through any suitable communication modality, including wired or wireless communication.

The controller 102 may comprise a non-transitory computer-accessible or computer-readable storage medium or storage 150 including instructions 160 stored thereon in a non-transitory form for operating a disinfection system and method according to the embodiments described herein. The instructions, when executed by the controller 102, can cause one or more processors 190 to carry out one or more of the steps described herein, in particular receiving one or more images or videos from the image capture devices 110, 120, one or more identification credentials from the identification module 125, one or more signals from the motion detector 115, and to utilize an artificial intelligence module 180 of the controller 102 to determine whether to operate the image capture devices 110, 120 in response to a signal from the motion detector 115, to identify a user based on the one or more credentials from the identification module 125, and/or to activate the light source 140.

The controller 102 may cooperate with the artificial intelligence module 180 to assess one or more frames of a captured video or image from the first and/or second image capture device 110, 120. The controller 102 may be configured to provide a compliance message to a user through the user interface 145. The compliance message may comprise a fail message including a notification of at least one step of a multi-step disinfection procedure, such as one or more of the 12 steps of the WHO handwashing procedure. The controller 102 may be configured to assess a numerical quality score of the disinfection procedure. The controller 102 may be configured to communicate the fail message via the user interface 145 if the numerical quality score is below a predetermined threshold. Alternatively, if the quality score is above the predetermined threshold, a pass or success message may be communicated via the user interface 145. The message may comprise a visual notification, such as a green flashing light for a successful procedure and a red flashing light for a failed procedure, words that appear on the user interface, one or more sounds corresponding to pass or fail, combinations thereof, or any other suitable manner of communicating pass or fail to the user.

In embodiments the controller 102 may be configured to indicate to a user that, if the procedure did not pass the quality assessment, which of the one or more steps of a required disinfection procedure, such as the WHO 12-step handwashing procedure, were problematic and should be improved when repeating the procedure. For example, the controller 102 may determine that the user did not properly rub palms with interlaced fingers for a sufficient amount of time. This may be indicated on the user interface 145, including with an animation or graphic illustrating the step that needs additional attention. In embodiments, the user interface 145 may be configured to guide the user through the handwashing or other disinfection process by showing an animation on a screen of the interface 145 for a duration during which a particular step should be continuously conducted, such as each step of the WHO 12-step handwashing procedure. This may advantageously engage the user throughout the procedure and encourage the user to follow exemplary techniques.

The controller 102 may be configured to require the user to repeat the handwashing process as many times as necessary to ensure a passing assessment. In alternative embodiments, the controller 102 may be configured to determine a threshold number of attempts before a user is considered by default to have passed the requirements, for example after three failed attempts. This advantageously educates each individual user about proper handwashing techniques while providing assurance for the employer that the users have properly disinfected before returning to or commencing work activities.

Upon determining a passing assessment, the controller 102 may automatically deactivate the image capture devices 110, 120. Upon determining a failed assessment, the controller 102 may continue to capture images or video using the image capture devices 110, 120 as the user is required to repeat the procedure.

The controller 102 may be configured to assess in a first instance of whether an image captured by the first and/or second image capture devices 110, 120 is capturing images of an activity of interest, for example handwashing or other disinfecting procedure as opposed to, for example, a user brushing their teeth, shaving, applying, or removing makeup, washing a dish or other object, or otherwise. The controller 102, upon determining that the activity is an activity of interest, may send a control signal to the first and/or second image capture devices 110, 120 to continue capturing images. By contrast, upon determining that the activity is not an activity of interest, the controller 102 may send a control signal to the first and/or second image capture devices 110, 120 to discontinue image capture. The controller 102 may be configured to re-assess whether the activity is an activity of interest at predetermined intervals throughout a duration in which a user remains proximate the motion detector, such as every half minute, every minute, every five minutes, or otherwise.

The controller 102 may cooperate with the artificial intelligence module 180 to analyze the captured images for improving the privacy and security of the image capture and assessment process. The controller 102 may be configured to identify and blur or remove static background features from at least one frame of a plurality of frames of a captured video. The controller 102 may also be configured to identify and blur or remove human faces from at least one frame of a plurality of frames of a captured video.

The controller 102 may be deployed completely on local devices or partially on local devices and partially on cloud resources or completely on cloud resources. In embodiments, the controller 102 may perform a portion of the analysis locally, such as to remove personally identifying or sensitive information from images, before transmitting the images or intermedia results using a communication modality to a cloud or remote location where one or more detection algorithms or models, including human keypoint or key area and/or hand or hand-finger detection, may be performed. In embodiments, the entirety of the system and method for disinfection is self-contained in a local device or location such that no data or images are transmitted; rather, the only interaction is between the system and an immediate user.

The artificial intelligence module 180 may comprise one or more computer vision algorithms configured to train and/or apply, for example, a machine learning model and/or a statistical algorithm for determining static features of video frames, human faces in video frames, and to assess the ideal configuration and duration of handwashing or other disinfecting poses for achieving satisfactory disinfection. The machine learning model and/or the statistical algorithm may be trained before the system 100A is deployed and/or may continue to be trained upon successive users of the system 100A. In embodiments, the machine learning model and/or the statistical algorithm may be customized by a user at a particular location based on the needs thereof.

The artificial intelligence module 180 may further advantageously obtain and/or determine a set of metrics generated from the disinfection procedure. The set of metrics may comprise in embodiments an amount of time spent on each step of the procedure. In the embodiment of handwashing in which a user is prompted to follow one or more of the 12 steps of the WHO-recommend procedure, the set of metrics may include a duration of each of the 12 steps. This may be determined, as discussed above, from a quantity of frames of a video feed in which a particular pose has been determined automatically from one or more keypoints or key areas of the user's body and/or hands and fingers. It will be appreciated that the 12-step WHO-recommend procedure is used by way of example only and it not the only disinfection procedure that can be monitored by the embodiments disclosed herein. Accordingly, the embodiments disclosed herein are not limited by the type of disinfection process or procedure being monitored.

The set of metrics may be used to train the artificial intelligence module 180 in advance or in substantial real-time to determine a duration and configuration of steps that can reliably lead to a passing assessment. For example, the set of metrics may be used to pretrain a model for detection of one or more steps of a disinfection procedure before the system is installed in a location or may be used to continually train and/or update the model as it is used in a location. In alternative embodiments, the captured video and images may be analyzed by one or more persons to determine the proper duration and configuration of steps. The analysis may be done in real-time or after a delay, and may be performed locally or remotely as suitable.

The artificial intelligence module 180 may identify and track a user, and particularly a target surface, such as the user's hands in a handwashing or other disinfection embodiment, using a computer vision modality, such as a facial recognition module, a pose estimation module, an object detection module, an objection recognition module, an object classification module, an object identification module, an object verification module, an object landmark detection module, an object segmentation module, a tracking module, or any other suitable modality. The computer vision modality may apply a bounding box around an identified person or object such as hands and/or a marker such as a virtual skeleton overlay superimposed onto the captured image of an identified person, such as over the user's hands, in particular the user's fingers. The bounding box may be applied based on automatically detected keypoints and/or key areas pertaining to a user's face, body, and/or hands, as suitable.

In embodiments where human keypoint or key area detection is combined with hand and finger detection, one or more keypoints or key areas pertaining to a user's body, including arm-related keypoints such as elbow, wrist, and hand tip may be detected in parallel with one or more keypoints or key areas pertaining to a user's hands, such as an elbow and a wrist keypoint or key area detected and analyzed in combination with all or some of the 22 hand-related keypoints or key areas noted above. This may advantageously allow for improved reliability of the system as frames of a video feed in which one or both of a user's hands are occluded, for example by the other hand, water and/or soap, or an object such as a faucet, may still be used to estimate a pose of a user as the keypoints or key areas of the arms, for example the elbow and wrist, may still be visible and used to estimate the pose of the hands.

In other words, the hand detection analysis may be anchored by the human keypoint or key area detection analysis, with human keypoint or key area detection supplementing and/or complementing the hand detection. The granularity of hand detection is combined with the reliability of human keypoint or key area detection.

For example, the system may determine a pose of a user's hands in one or more frames in which one or both of the hands is substantially occluded by comparing a position of one or more keypoints or key areas on the user's arms relative to the position of the one or more keypoints or key areas on the user's arms in adjacent frames in which the user's hands are not occluded, and the pose of the user's hands is readily detectable using hand detection modalities described herein. This increases the number of frames that are available for assessing a duration and amplitude of a pose of a disinfection procedure as frames in which the user's hands are occluded need not be discarded or ignored. The reliability of a system according to embodiments of the present disclosure is hereby improved due to a higher granularity of data from which the poses are estimated.

In embodiments, the facial recognition module and/or object recognition and classification modules may be used to identify and blur or remove static features and/or human faces or any other suitable parts of an image. In embodiments, everything except for a target surface of the disinfection procedure, such as a user's hands and optionally part or an entirety of the user's arms and/or torso, may be automatically removed from the images or blurred. The system may advantageously store the captured images and/or videos on the storage medium in the edited form, thereby excluding that any permanent image of a user's face or any other private or sensitive imagery is permanently stored on or accessible through the system.

In embodiments, skeleton markers defining a virtual skeleton overlay comprising, for example, one or more keypoints or key areas and one or more connecting lines representing body segments may be applied onto the image or frame when a person is detected, and one or more bounding boxes or classes may be applied onto the image or frame for identified objects. The bounding boxes may comprise a point, width, and/or height. The disinfection system may further be configured to provide a label that specifies an identified class of an identified object and data specifying where the identified object appears in an image. For example, a label may identify the hands as belonging to a particular user, as the user's face and static features from the video may have been removed by the artificial intelligence modality. The virtual skeleton overlay may define or cooperate with a human pose skeleton.

The virtual skeleton overlay may comprise one or more keypoints or key areas which may be one or more features of the identified person, such as joint keypoints or key areas corresponding to an identified joint, such as a wrist joint, one or more metacarpophalangeal joints, one or more distal interphalangeal joints, fingertip keypoints or key areas, facial keypoints or key areas, combinations thereof, or otherwise.

The virtual skeleton overlay may further comprise one or more body segments extending between one or more of the keypoints or key areas. The body segments may extend along the fingers, such as along the metacarpals and/or phalanges between the wrist joint, metacarpophalangeal joints, and the interphalangeal joints. It will be appreciated that the artificial intelligence module may be configured to perform an assessment on more than the hands, and may perform a virtual skeleton overlay on, for example, the arms, body, legs, and head of a person while also blurring in embodiments the user's face and any other identifying features, such as tattoos or other distinctive features. It will also be appreciated that the body segments extending between detected keypoints and/or key areas may be, in embodiments, merely artificial and exterior to the detection of keypoints or key areas, and provision of such connections may advantageously help visualize the detection, for example as a user reviews the annotation and/or analysis of one or more captured frames of a video.

The computer vision modality of the artificial intelligence module may perform detection-based tracking across one or more frames of the captured video and/or images, performing in embodiments a finger motion pattern. The analysis may comprise evaluation at each step (for instance, each step of the WHO 12-step handwashing procedure) that assesses strengths patterns including a combination of duration and amplitude. The artificial intelligence module may leverage data-driven deep learning for finger detection. The tracking algorithm used by the artificial intelligence module may track individual hands using multi-object tracking considering clutter and misdetection. The model, which may be a machine learning model, may be trained using automated feature selection. The fingers may be detected simultaneously and/or as part of a same hand, this allowing for multiple object tracking of fingers and/or hands and/or arms. This further allows the system to determine which hands the fingers correspond to, and which arms the hands correspond to.

In embodiments, finger and finger-motion detection includes keypoint or key area detection. For example, the system may utilize a pretrained model for hand-related keypoint or key area detection involving a predetermined number of keypoints or key areas pertaining to the hand, such as but not limited to 1) a wrist keypoint or key area, 2) a scaphoid keypoint or key area, 3) a trapezium keypoint or key area, 4) a first metacarpal keypoint or key area, 5) a first proximal phalange keypoint or key area, 6) a thumb tip keypoint or key area, 7) a second metacarpal keypoint or key area, 8) a second proximal phalange keypoint or key area, 9) a second middle phalange keypoint or key area, 10) an index finger tip keypoint or key area, 11) a third metacarpal keypoint or key area, 12) a third proximal phalange keypoint or key area, 13) a third middle phalange keypoint or key area, 14) a middle finger tip keypoint or key area, 15) a fourth metacarpal keypoint or key area, 16) a fourth proximal phalange keypoint or key area, 17) a fourth middle phalange keypoint or key area, 18) a ring finger tip keypoint or key area, 19) a fifth metacarpal keypoint or key area, 20) a fifth proximal phalange keypoint or key area, 21) a fifth middle phalange keypoint or key area, and 22) a pinkie finger tip keypoint or key area. While the above keypoint or key areas pertaining to one or both of a user's hands have been described, it will be appreciated that the above embodiment is exemplary, and any suitable number, combination, and use of hand-related keypoints or key areas may be used.

The human disinfection system may utilize a direct regression-based framework to identify and apply the one or more keypoints or key areas, a heatmap-based framework, a top-down approach, a bottom-up approach, a combination thereof, or any other suitable approach for identifying the keypoints or key areas. A direct regression-based framework may involve the use of a cascaded deep neural network (DNN) regressor, a self-correcting model, compositional pose regression, a combination thereof, or any other suitable model. A heatmap-based framework may involve the use of a deep convolutional neural network (DCNN), conditional generative adversarial networks (GAN), convolutional pose machines, a stacked hourglass network structure, a combination thereof, or any other suitable approach. In embodiments, direct regression-based and/or heatmap-based frameworks may make use of intermediate supervision.

In embodiments, a heatmap-based approach outputs a probability distribution about each keypoint or key area using a DNN from which one or more heatmaps indicating a location confidence of a keypoint or key area are detected. The location confidence pertains to the confidence that the joint or other feature is at each pixel. The DNN may run an image through multiple resolution banks in parallel to capture features at a plurality of scales. In other embodiments, a key area may be detected, the key area corresponding generally to an area such as the elbow, knee, ankle, etc.

A top-down approach may utilize a suitable deep-learning based approach including a face-based body detection for human detection, denoted for example by a bounding box from or in which keypoints or key areas are detected using a multi-stage cascade DNN-based joint coordinate regressor, for example. A “top-down approach,” as defined herein, indicates generally a method of identifying humans first and then detecting keypoints or key areas of the detected humans.

A bottom-up approach may utilize a suitable keypoint or key area detection of body parts in an image or frame, which may make use of heatmaps, part affinity fields (PAFs), or otherwise. After identifying keypoints or key areas, the keypoints or key areas are grouped together, and persons are identified based on the groupings of keypoints or key areas. A “bottom-up approach,” as defined herein, indicates generally a method of identifying keypoints or key areas first and then detecting humans from the keypoints or key areas.

The system 100A may utilize two categories of keypoints or key areas with separate models utilized by the processor 111 for each category. A first category of keypoints or key areas may include keypoints or key areas automatically generated by a suitable model as described above, such as a machine learning model. In embodiments, the first category of keypoints or key areas are semantic keypoints identified by a first model, such as a deep learning method, for example Mask RCNN, PifPaf, or any other suitable model. The keypoints or key areas automatically generated for the first category may include a nose keypoint or key area, a left eye keypoint or key area, a right eye keypoint or key area, a left ear keypoint or key area, a right ear keypoint or key area, a left shoulder keypoint or key area, a right shoulder keypoint or key area, a left elbow keypoint or key area, a right elbow keypoint or key area, a left wrist keypoint or key area, a right wrist keypoint or key area, a left hip keypoint or key area, a right hip keypoint or key area, a left knee keypoint or key area, a right knee keypoint or key area, a left ankle keypoint or key area, a right ankle keypoint or key area, combinations thereof, or any other suitable keypoint or key area.

A second category of keypoints or key areas may include predicted keypoints or key areas obtained or derived from the first category of keypoints or key areas using geometric prediction, such as a head top keypoint or key area, a right handtip keypoint or key area, a left handtip keypoint or key area, a chin keypoint or key area, a left foot keypoint or key area, a right foot keypoint or key area, combinations thereof, or other keypoints or key areas, optionally using a second suitable model and based on the first category of automatically generated keypoints or key areas. In embodiments, the second category of keypoints may be interest points, and may be determined by a same model as the first category or a distinct, second model, which may include one or more of a self-supervised learning model such as Moco, SimCLR, or any other suitable model. The second model may be configured to determine the second category of keypoints as a function of and/or subsequent to detection of the first category of keypoints.

Advantageously, the artificial intelligence module of the present disclosure only requires in embodiments a view of the user's hands and fingers, as the hands and fingers can be identified from static features and from the rest of the user's body. Thus, a user who is wearing long-sleeved clothing while performing the disinfection procedure can still be assessed, whereas in many existing attempts to provide handwashing analysis the arms must be identified in addition to the hands and fingers.

Further, the artificial intelligence module of the present disclosure can conduct an assessment of single-hand disinfection activity, which makes the system applicable for users who have only one hand and for a number of disinfection activities that involve a user disinfecting a surface or object using primarily one hand, i.e., their dominant hand. In embodiments, an object-detection module may be utilized in parallel with, prior to, subsequent to, or in any suitable form of cooperation with a hand-detection and/or human keypoint detection model. The system may be pretrained or trained to recognize specific objects, a particular class of objects, labels, and/or particular actions.

A general object may include a class of objects, such as restaurant objects generally, medical-related objects generally, clothing generally, laboratory-related objects, or any other object. In embodiments, the system is configured to extend keypoint or key area detection to a plurality of objects. The system may be configured to allow a user to use a pretrained model or to train the system to recognize a general class of objects. This may be done, in embodiments, by “showing” the system the general object in one or more angles, by holding and manipulating the object within the field of view of one or more cameras of the system and/or in one or more different locations and/or in one or more different activities. The system may also utilize one or more images uploaded of the general object class and/or may cooperate with a suitable object detection model that may be uploaded to the system.

A specific object may include any suitable object that is specific to a user. For example, a restaurant franchisee may want the system to recognize a particular type of dish or cooking implement but not dishes generally. The system may be configured to be trained by a user to recognize one or more specific objects, for example by prompting the user through a user interface to hold and/or rotate the object within a field of view of one or more cameras so that the system may learn to recognize the specific object and/or different uses of the object.

A specific object may include a medical device, such as a face shield, surgical forceps, endoscope, stethoscope, or otherwise. In embodiments, one or more keypoints or key areas on the object may be specified. The presenter or viewer may apply markings onto areas of the surface of the object before placing the object in the field of view of the camera so as to train the system to identify the markings as keypoints or key areas. In other embodiments, the presenter or viewer may annotate one or more frames of a captured video or image to denote the keypoints or key areas of the object and/or bounding boxes corresponding to the keypoints or key areas and the object of interest. This allows the system to extract features of interest for accurate and automatic detection of the object when pertinent.

In embodiments, a presenter may train the system to recognize a plurality of specific items, such as medical devices pertaining to a particular procedure and/or operating room as opposed to medical devices generally. The system may then automatically extend detection to the specific items when the items appear within the field of view of the image capture device. In embodiments, the user may determine one or more custom, user-specific modes of operation between which the user may toggle, such as to specify a mode in which one or more objects are automatically detected by extending keypoint or key area detection to the one or more objects and evaluating a disinfection procedure thereof and/or a mode in which the one or more objects are not included in the cropped image, i.e., ignored.

The system may likewise be configured to recognize a particular label (such as a barcode, a QR code, an Aruco code, plain text, or any other suitable label) by uploading the label through a user interface or by arranging the field of view of one or more cameras of the system to capture the label (such a label placed on or adhered to an object surface) such that the system may learn to recognize such labels. In embodiments, the system is configured to extend keypoint or key area detection beyond one or more presenters and to include one or a combination of a label, a specific object, and a general object.

It will be appreciated that while the method for assessing disinfection quality has been described, other embodiments are within the scope of the disclosure, including other artificial intelligence modalities and approaches for quantifying quality of handwashing and/or ensuring privacy and eliminating redundant and/or irrelevant features. This advantageously reduces the time and labor involved with manually evaluating handwashing quality (which is limited by subjectivity) and inherently improves on privacy for individual users, particularly as disinfection procedures are frequently carried out in restrooms both private and public.

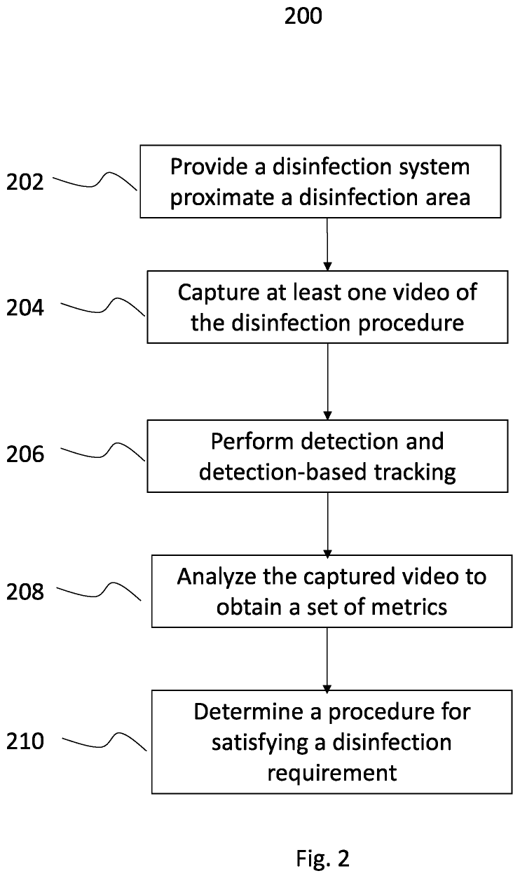

A disinfection monitoring method 200 according to an embodiment is shown in FIG. 2. The method 200 may include one or more of the following steps in any order or number along with any other suitable steps. A first step 202 includes providing a disinfection monitoring system according to the embodiments proximate a disinfection area. A disinfection area may include a sink in a restroom or in a prep area, such as in a food preparation facility or in a healthcare facility. The system may be provided with certain local components, such as at least one image capture device, identification module, motion detector, user interface, and other components as described herein.

A second step 204 includes a step of capturing at least one image and/or video of the disinfection procedure using at least the first image capture device 110. As described previously, the user may be guided through the user interface 145 to perform one or more predetermined actions or poses optionally for a predetermined duration. For instance, the user interface 145 may provide indicia or animations that show the user exemplary technique for the one or more poses. While visible-light video has been described, it will be appreciated that video of any suitable wavelength of light, such as infrared or near-infrared, may be captured alternatively or additionally as suitable.

A third step 206 includes a step of performing detection and detection-based tracking of the user's fingers and/or one or both hands from the images obtained from the image and/or video captured in step 204. The detection and detection-based tracking may be performed by an artificial intelligence modality and/or processor of a controller of the system. In embodiments, the detection and detection-based tracking comprise keypoint and/or key area detection as disclosed herein.

A fourth step 208 of analyzing using an artificial intelligence modality and/or processor of a controller of the system to obtain a set of metrics regarding the disinfection procedure. In embodiments, the set of metrics may include a duration spent on each step, which may be identified from the videos using one or more computer vision techniques, such as a machine learning model or statistical algorithm.

A fifth step 210 may include a step of determining, from the images obtained from the image and/or video captured in step 204, a configuration of the disinfection procedure which satisfies a disinfection requirement. In embodiments, the disinfection requirement may be score for each step of the disinfection procedure, such as each the 12 steps of the 12-step WHO handwashing procedure. The controller 102 may be configured to communicate the fail message via the user interface 145 if the score for a given step is below a predetermined threshold. Alternatively, if the score for a given step is above the predetermined threshold, a pass or success message may be communicated via the user interface 145.

The fifth step 210 may be advantageous not only regarding handwashing, regarding which there is substantial disagreement as to the proper steps, order of steps, and duration of steps, but also regarding other disinfection procedures regarding which there may not be a consensus regarding “best practices” or quantifiable observations regarding quality of procedures. This may include without limitation the disinfection of various pieces of equipment, such as medical or dental equipment or food preparation-related equipment, the disinfection of other parts of the human body, the disinfection of spaces such as a kitchen, operating room, or otherwise.

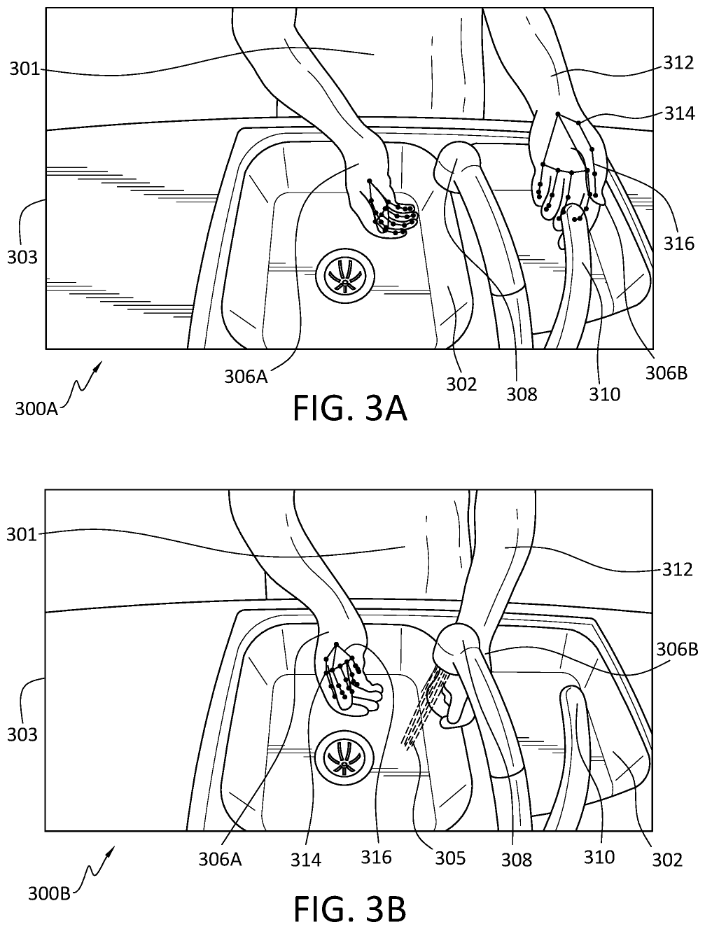

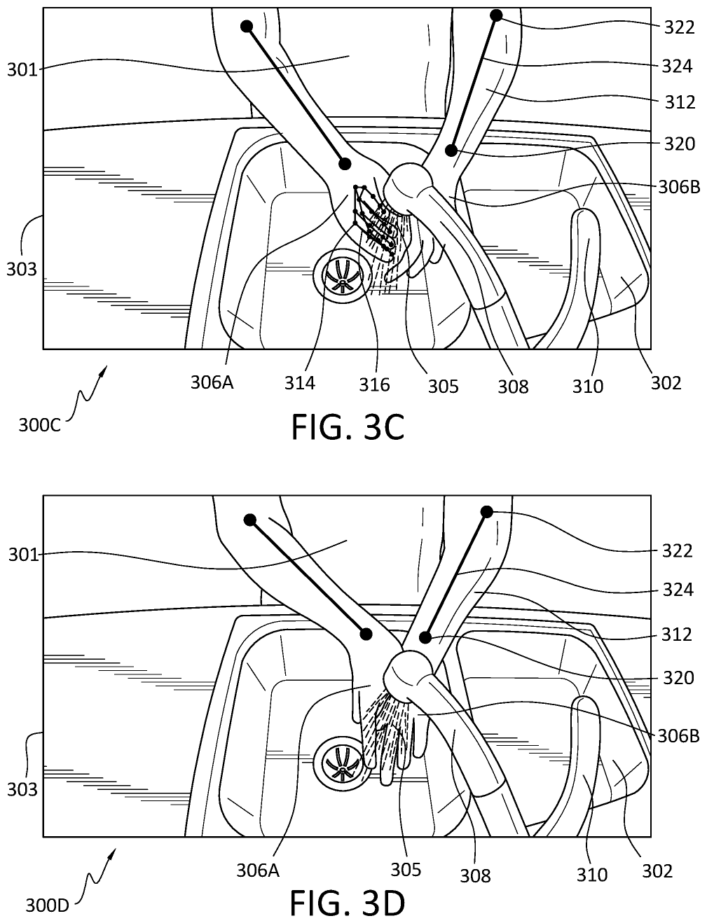

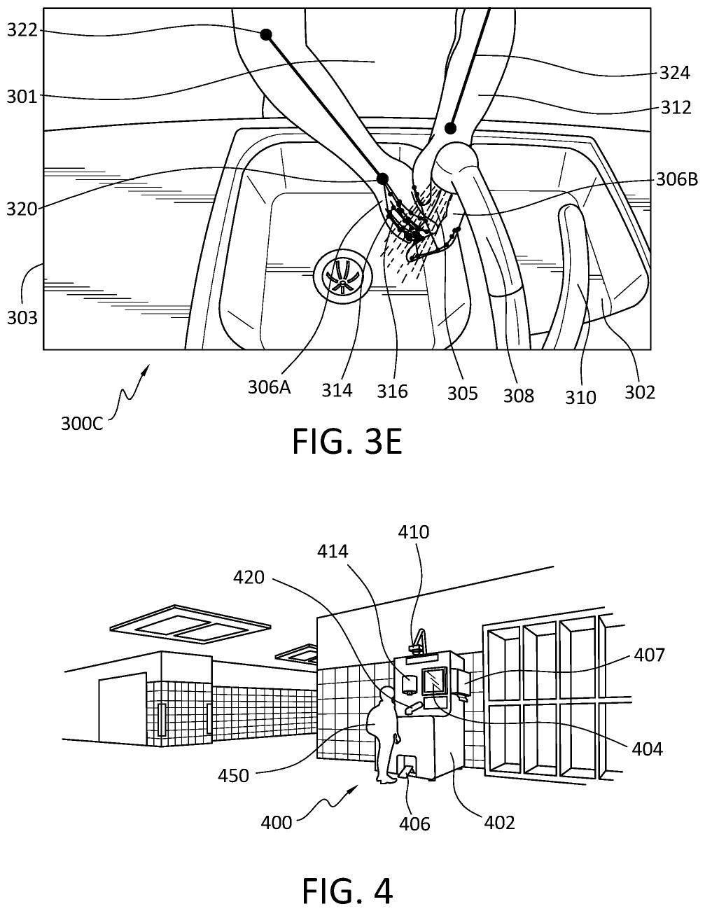

Turning to FIG. 3A, an annotated image 300 generated by a disinfection monitoring system according to an embodiment of the disclosure is shown and described. The annotated image 300 may capture a user 301 within a field of view 303 of a suitable image capture device as described above. The field of view 303 may be oriented such that one or both of a user's hands 306A, 306B and/or arms 312 are captured in the annotated image 300. The field of view 303 may be oriented further such that a region of interest for the disinfection system, such as a wash basin 302, may be captured. The wash basin 302 may comprise one or more structures such as a faucet 308 and/or a handle 310. The field of view 303 may represent an entire field of view captured by a system, or may correspond to one image capture device of a plurality of image capture devices. For example, as described above, a distinct image capture device having a different orientation may be provided to capture the user's face and/or uniform for identification purposes.

As a user approaches the wash basin 302 to perform a disinfection procedure, the system may capture a video feed comprising a plurality of frames, of which the annotated image 300 may be one. The system utilizes a suitable artificial intelligence modality as described herein to detect one or more poses of a disinfection procedure and to determine a score thereof. For instance, the system may utilize hand detection to determine one or more keypoints or key areas 314 of one or both of the user's hands 306A, 306B. The system may determine any suitable number of keypoints or key areas 314 corresponding to any suitable features of the hand.

Each keypoint or key area may be connected to or associated with a proximate keypoint or key area. The connections 316 between keypoints or key areas 314 may be omitted in embodiments, with the determination of the pose of a disinfection procedure conducted on the basis of the keypoints or key areas without consideration of or overlaying a connecting line between keypoints or key areas. Such connections and connecting lines may be, in embodiments, merely artificial and external to the detection of keypoints and key areas, and provision of such connections may advantageously help visualize the detection, for example as a user reviews the performance of the system.

Turning to FIG. 3B, an annotated image 300B, having the field of view 303 and capturing the user 301, is shown. The annotated image 300B may be a frame from the same video feed as the annotated image 300A and captured subsequently thereto. The annotated image 300B comprises the keypoints or key areas 314 and connections 316 as detected on one of the hands 306A, but not on the other hand 306B, which is occluded by the faucet 308 and a stream of water 305.

Turning to FIG. 3C, an annotated image 300C, having the field of view 303 and capturing the user 301, is shown. The annotated image 300C may be a frame from the same video feed as the annotated images 300A, 300B, and captured prior, simultaneously, or subsequently thereto. The annotated image 300C comprises the keypoints or key areas 314 and the connections 316 as detected on one of the hands 306A, but not on the other hand 306B, which remains occluded by the faucet 308 and the stream of water 305. The system may be configured to determine one or more keypoints or key areas 320, 322 for human keypoint detection in combination with or in parallel with hand detection corresponding to the keypoints or key areas 314. The keypoints or key areas 320, 322 may correspond respectively to a wrist keypoint or key area and an elbow keypoint or key area and may remain visible to the image capture device, unoccluded by the faucet 308, handle 310, or the stream of water 305, when the hand 306B is occluded.

Turning to FIG. 3D, an annotated image 300D, having the field of view 303 and capturing the user 301, is shown. The annotated image 300D may be a frame from the same video feed as the annotated images 300A, 300B, 300C and captured prior, simultaneously, or subsequently thereto. The annotated image 300D comprises the keypoints or key areas 320, 322 as detected on the user's arms. The user's hands 306A, 306B are occluded in FIG. 3D from detection by, e.g., the faucet 308, the handle 310, the stream of water 305, and each other. The provision of the disinfection system advantageously facilitates human keypoint detection such that a pose corresponding to FIG. 3D may be extrapolated from the wrist keypoints or key areas 320 and/or the elbow key areas 322.

Turning to FIG. 3E, an annotated image 300E, having the field of view 303 and capturing the user 301, is shown. The annotated image 300E may be a frame from the same video feed as the annotated images 300A, 300B, 300C, 300D and captured prior, simultaneously, or subsequently thereto. The annotated image 300D comprises the keypoints or key areas 320, 322 as detected on the user's arms. The user's hands 306A, 306B are partially occluded in FIG. 3E from detection by, e.g., the faucet 308, the handle 310, the stream of water 305, and each other. The annotated image 300E comprises a plurality of keypoints or key areas 320, 322, 314 pertaining to both human key keypoints or key areas and hand keypoints or key areas, in FIG. 3E pertaining to both of the hands 306A, 306B and both of the arms 312.

As seen in FIGS. 3A-3E, the annotated images 300A-300E may comprise one, a plurality, or an entirety of a predetermined number of keypoints or key areas of a hand. The system may detect a different number and combination of keypoints or key areas in each hand and/or in each frame.

The system may advantageously be configured to associate a particular pattern of keypoints or key areas of one or both hands with a particular pose of a disinfection procedure, for example, a palm to palm pose, a backs of fingers to opposing palms with fingers interlocked pose, a palm to palm with fingers interlaced pose, a right palm over left dorsum with interlaced fingers pose, a rotational rubbing of thumb clasped in the opposite palm pose, and so forth. The hand detection model may be configured to cooperate with a distinct model trained to associate predefined poses with detected patterns of keypoints or key areas of a hand and/or a user's body within a single frame or across a plurality of frames of a video feed.

As discussed above, the utilization of human keypoint or key area detection in parallel and/or in combination with hand detection advantageously results in a more-robust detection of a pose of a disinfection procedure from the hand and its associated keypoints or key areas and/or the human keypoints or key areas. In frames of a video feed in which one or both hands is occluded as in FIGS. 3B and 3C above, the detection of human keypoints or key areas pertaining to the arm may be used to extrapolate a determination of a pose of a disinfection procedure when hand-related keypoints or key areas are undetectable, making more frames of a video feed available for analysis. This increases the granularity of the analysis and the reliability of the detection of poses.

While the use of a combination of human keypoint or key area detection and hand or hand-finger detection has been described, it will be appreciated that human keypoint or key area detection alone may be used, or hand or hand-finger detection alone may be used for one or more frames of a video feed. In embodiments, the system may alternate between using human keypoint or key area detection for a predetermined or automatically determined number of frames of a video feed and hand or hand-finger detection for a predetermined or automatically determined number of frames of a video feed, as suitable.

It has been surprisingly found that the combination of human keypoint or key area detection and hand or hand-finger detection provides improved results relative to either human keypoint or key area detection alone or hand or hand-finger detection alone, as in many activities and for many video feeds capturing said activities, neither human keypoint or key area detection alone, hand or hand-finger detection alone, is 100% accurate. By providing a system configured for using a combination of human keypoint or key area detection and hand or hand-finger detection, the inaccuracies or errors of either detection method may be mitigated to provide a more-accurate overall detection.

In embodiments, the system may utilize sensor fusion principles and/or Bayesian filters, such as but not limited to particle filters and/or Kalman filters, to properly combine the aforementioned detection methods. The use of Bayesian filters advantageously allows the system to perform multiple-object tracking, for example tracking both arms, hands, and the fingers, and/or any suitable objects upon which a disinfection procedure is to be performed. For example, a suitable Bayesian filter such as a particle filer or a Kalman filter may be provided and used to eliminate noise from one or both of the detection methods.

The Bayesian filter, in embodiments a particle filter or a Kalman filter, generates estimates of the state of, for example, the objects of interest, in embodiments the hands and/or fingers, by utilizing both current observations and a previous prediction for one or more frames of a video feed. The predictions may be represented or computed as any suitable distribution, in embodiments as Gaussians comprising, for example, a normal distribution of probable locations and/or positions of the hands, arms, and/or fingers, by predicting the probable location and/or positions of a bounding box for one or both hands and/or keypoints or key areas. While Gaussians have been described, it will be appreciated that any suitable distribution may be determined or utilized, such as a multi-peak distribution.

The Kalman filter may, in embodiments, be used to estimate or improve an estimation of one or both of a state and an uncertainty of a detection of the hands and/or fingers for a particular frame of a video feed to provide continuity, such as to check the model results at predetermined intervals compared to a previous prediction. For example, the system may apply the Kalman filter to generate a prediction of the state and uncertainty of a detection, i.e., a position, of the hands and/or fingers for a frame, particularly a future frame, and then update and/or correct the prediction using a difference between an actual measurement and prediction. The prediction may be generated for, in embodiments, an immediately subsequent frame. The prediction may be generated for a location of a bounding box surrounding the hand and/or fingers, for a particular keypoint or key area of the hand and/or fingers, or for any other suitable object or location.

The Kalman filter may utilize models for predicting a future location of the bounding box and/or keypoint or key area by utilizing one or more models of, in embodiments, position, constant velocity, and/or acceleration of the bounding box and/or keypoint or key area.

This facilitates the generation and validation of predictions in substantially real time. In embodiments the system may apply the Kalman filter between the human keypoint or key area detection and hand or hand-finger detection to validate the predicted location of the hands and/or fingers in a particular frame or frames. That is, the Kalman filter may be used such that a predicted state generated by human keypoint, or key area detection is validated against a measurement using the hand or hand-finger detection model, and vice versa. In embodiments the predicted state of the hand and/or fingers based on human keypoint and/or key area detection may be validated additionally or alternatively against a measurement from the human keypoint and/or key area detection model, and likewise for the hand or hand-finger detection model.

The Kalman filter may further utilize, in embodiments, Extended Kalman filters and/or unscented Kalman filters for approximating the position and velocity of the hands and/or fingers. For example, the system may utilize an Extended Kalman filter configured for state prediction and estimation of a non-linear system model, in embodiments by linearizing the process model for each frame by computing the Jacobian of a transition matrix about the state vector.

The Kalman filter may utilize a weighted sum, a winner-take-all, and/or other suitable technique to combine and sort information obtained using a combination of human keypoint or key area and hand or hand-finger detection methods and corresponding models. For example, the system may fuse the data from the different approaches by taking a weighted sum of the Kalman-filtered bounding boxes and/or keypoints or key areas detected regarding a user's hands, fingers, and/or arms, or may adopt a filtered bounding box or keypoint or key area with a highest probability or value.

In embodiments in which a weighted-sum technique is used, any suitable weight may be applied to the human keypoint or key area detection relative to the hand or hand-finger detection results, for example equal weights or different weights as suitable. The weights may be determined dynamically based on each frame; for instance, upon determining that one or more features of one or both hands is occluded in a frame, greater weight may be given to human keypoint or key area detection relative to hand or hand-finger detection, and vice versa. In embodiments in which a winner-take-all technique is used, only the human keypoint or key area detection is used when one or more features of one or both hands is occluded in one or more frames. Similarly, only hand or hand-finger detection data may be used when one or more features of one or both arms is occluded in one or more frames.

In embodiments, the techniques described above apply to a plurality of image capture devices configured to capture a plurality of images of the user's hands and arms. In embodiments, the image capture devices may be configured to capture the plurality of images from different angles and/or positions. As a user washes their hands or an object within the field of view of one or more of the image capture devices, the system may be configured to perform keypoint or key area detection or hand or hand-finger detection on the images obtained from each of the image capture devices, with the data and detection performed independently on the images from different cameras being fused as described herein. For example, the system may be configured to utilize a Bayesian filter such as a Kalman filter to reconcile the data and to select one or both of the detection results as suitable, using in embodiments a weighted-sum or winner-take-all technique as suitable. When one or both hands is occluded in the field of view of one of the image capture devices, for instance, greater weight may be automatically given to the detection from a different image capture device.

While a Kalman filter has been described specifically, it will be appreciated that any suitable filtering method, such as Bayesian filters generally, may be used, and the present disclosure is not limited to the above-described Kalman filters and use thereof, but rather extends to any suitable method for filtering, fusing, and otherwise processing, interpreting, and utilizing information regarding a user of the disinfection system.

FIG. 4 shows a disinfection system 400 according to an embodiment of the present disclosure. The system 400 may include or define a housing 402 in which one or more of the components described herein may be housed and accessed. For example, the housing 402 may define a user interface 404 which may comprise a screen, such as a touch screen, that displays instructions for handwashing and/or handwashing analysis and results. For example, the user interface 404 may provide step-by-step instructions with real-time feedback on each step of a procedure, such as the 12-step WHO handwashing procedure.

A user 450 may initiate a handwashing procedure by utilizing a foot pedal 406, which may be configured to initiate a flow of water. Soap and/or UV-visible material may be dispensed at a dispenser 414, which may comprise a sensor configured for facilitating automatic dispensing. Water may be provided at a faucet 420. The housing 402 may also support or define a dispenser 406 for paper towels. The dispenser 406 may additionally or alternatively support an air-drying modality.

The system 400 includes one or more image capture devices 410 arranged at any suitable location and angle for capturing the hands and/or arms of the user 450 and/or identification credentials, such as the user's face and/or an ID badge, in embodiments. As discussed above, the system 400 may include a plurality of image capture devices arranged so as to capture different fields of view and/or different angles of the user's hands or other objects, so as to mitigate detection challenges arising from occlusion of one or both of the hands and arms from one of the image capture devices during one or more steps of a disinfection procedure, such as handwashing.

A disinfection data collection system and method are designed to help to properly collect the data for training the real time monitoring system. The goal of data collection is to obtain videos or images of a disinfection process or procedure and a corresponding ground-truth score of the overall disinfection quality or disinfection quality for each step of the disinfection process. The data collection may utilize the same techniques as the real time monitoring system to protect privacy, trigger recording automatically, and filter out the irrelevant activities. The ground-truth score could be determined by human experts or from other sensing/computer vision techniques such as image analysis by the disinfection data collection system.

FIG. 1B is a diagram of a disinfection data collection system 100B according to an embodiment of the present disclosure that may be used in particular to collect data that may be used to train the AI modules 180. As shown in FIG. 1B, the disinfection data collection system 100B includes many of the same elements as the real time disinfection monitoring system 100A. Accordingly, the elements of the disinfection data collection system 100B that were discussed previously with respect to FIG. 1A may not be discussed in again in relation to FIG. 1B. It will be initially noted that although an identification module is not shown as part of the disinfection data collection system 100B, such module may optionally be included as circumstances warrant. It will also be appreciated that in some embodiments a single disinfectant system may include the elements of both the system 100A and the system 100B if desired. Thus, it will be appreciated that the division of the disinfectant system 100 into system 100A and 100B is for ease of explanation, but does not imply that a single disinfectant system cannot be implemented with the elements of both the system 100A and the system 100B.

In the embodiment of the system 100B, the second image capture device 120 may be or comprise any suitable image capture modality, including a digital camera. In particular, the second image capture device may be specially configured for capturing an image of an object to be disinfected, such as a user's hands, under UV light. Accordingly, the camera 120 may be a camera comprising a UV filter configured to allow ultraviolet light to pass and to absorb or block all visible and infrared light. In embodiments, a lens of the camera 120 may comprise or be formed from fused quartz, quartz, and/or fluorite so as to allow a desired wavelength or range of wavelengths of UV light to pass.

The second image capture device 120 may comprise an attachment component configured to attach the second image capture device 120 to any suitable surface, such as a wall, ceiling, bathroom fixture, or otherwise, and may comprise one or more lockable joints allowing the direction of the camera 120 to be adjusted. In embodiments the light source 140 may be a UV light configured to provide UV light toward the object or objects being disinfected. The UV light 140 may provide UV light in the direction of a sink where a user is washing their hands, for example.

In embodiments, the system 100B may comprise or cooperate with at least one UV-active material 105. The UV-active material 105 may be dispensed through a dispensing unit upon receiving an activation signal from the controller 102. The dispensing unit may be configured to dispense a predetermined amount of UV-active material 105 for each instance of a disinfecting procedure, such as handwashing. The UV-active material 105 may be any suitable material that reacts in a suitable way to a particular wavelength or range of wavelengths of light, such as from the light source 140. The UV-active material 105 may be a fluorescent material, a phosphorescent material, or otherwise, and may include a powder, such as a doped or undoped strontium aluminate powder, zinc sulfide power, avobenzone, cyanines such as indocyanine, fluorophores such as fluorescein, or any other suitable compound. The UV-active material 105 may be primarily liquid, a gel, or primarily a solid powder, as suitable, and may be mixed with a soap substance or may be administered separately from a soap dispenser.

In embodiments, the data collection system 100B may comprise an image analysis module 185 as a AI module for the system. The image analysis module, which is a type of the computer vision modality discussed previously. In operation, the image analysis module 185 is configured to analyze each image so that the ground-truth score may be obtained.

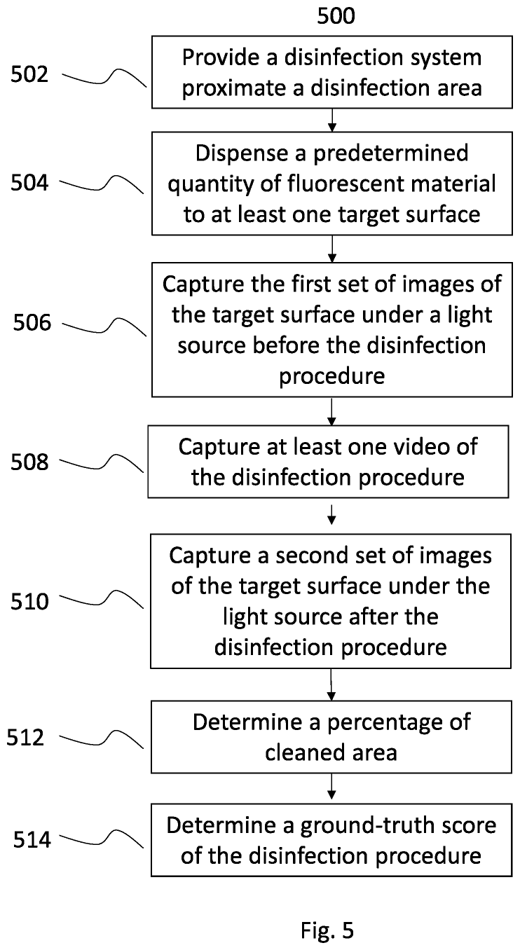

A disinfection data collection method using a UV glowing technique 500 according to an embodiment is shown in FIG. 5. The method 500 may include one or more of the following steps in any order or number along with any other suitable steps. A first step 502 includes providing a disinfection data collection system according to the embodiments proximate a disinfection area. A disinfection area may include a sink in a restroom or in a prep area, such as in a food preparation facility or in a healthcare facility. The system may be provided with certain local components, such as at least one image capture device, identification module, motion detector, user interface, and other components as described herein.

A second step 504 includes dispensing a predetermined quantity of UV-active material to at least one target surface for disinfection. In handwashing embodiments using a system 100B as described above, the UV-active material may be a powder, liquid, gel, or other substance that may be dispensed to a user along with soap or separately from soap, and may be dispensed using a conventional soap-dispensing unit. The soap-dispensing unit may be configured with an actuator or valve that is configured to receive a control signal from a controller to dispense a predetermined amount of UV-active material to each user, the consistent amount of UV-active material between users serving to provide for improved analysis of the efficacy of the disinfection procedure. A user interface of the system 100B may be configured to instruct the user to spread the UV-active material evenly about the target surface, i.e., their hands.

A third step 506 includes a step of capturing a first set of images of the target surface under a light source prior to the disinfection procedure. The light source may be a UV light configured to specially illuminate the target surface upon which the UV-active material has been applied and evenly spread in the second step 504. At least one image capture device of the system 100B may be configured to capture UV-specific images, for example by comprising one or more UV filters or UV lenses.

A fourth step 508 includes a step of capturing at least one video of the disinfection procedure following the application of the UV-active material. The at least one video may be captured by a second image capture device of the system 100B, for example a camera configured to capture visible-light video as opposed to UV-specific images. As described previously, the user may be guided through the user interface to perform one or more predetermined actions or poses optionally for a predetermined duration. For instance, the user interface may provide indicia or animations that show the user exemplary technique for the one or more poses. While visible-light video has been described, it will be appreciated that video of any suitable wavelength of light, such as infrared or near-infrared, may be captured alternatively or additionally as suitable.

A fifth step 510 includes a step of capturing a second set of images of the target surface under the light source after the disinfection procedure has concluded. A pose of the target surface such as the user's hands under the light source may be substantially the same as the pose utilized in the first image captured before the disinfection procedure. It will be appreciated that more than one image of the target surface may be captured at one or both of steps 506, 510. For example, images corresponding to different poses, such that a substantial entirety of the user's hands are imaged, may be captured.

A sixth step 512 includes a step of determining from the images obtained the steps 506, 510 percentages of each part of one or more predetermined parts of a user, such as the fingers, of the target surface that has been cleaned. The sixth step 512 may be performed by the image analysis module 185. In embodiments, the image analysis module 185 comprises a computer vision module comprising for example a machine learning model, a statistical algorithm, or any other suitable modality. The predetermined parts may include segments or zones of the user's fingers corresponding to, in embodiments, the distal, middle, and proximal phalanges and the metacarpal of one or more fingers of each hand. The system may be configured to assess at the sixth step 512 whether, which, and to what extent (such as by percentage) one or more of the zones of the user's fingers has UV-active material remaining.

While UV-active material and UV light have been described, it will be appreciated that any suitable alternative may be used, including the use of fluorescent, phosphorescent, or other materials having a desired reactivity to light of any particular wavelength or range of wavelengths.

This advantageously allows the system to determine automatically, based on the still-contaminated zones, which step or steps of a disinfection procedure the user should be prompted to repeat and optionally for how long. For example, a user may be prompted, upon the system determining at the sixth step 512 that the zones corresponding to the distal phalanges of the right hand are still contaminated, that the user should repeat step 8): rotational rubbing, backwards and forwards with clasped fingers of right hand in left palm, for 10 seconds. Any suitable duration and combination of steps may be prescribed based on the extent of contamination remaining on particular zones.

A seventh step 514 may calculate a ground-truth score of each predetermined part or area of a user's fingers and/or one or both hands and an overall score of the whole disinfection procedure. In embodiments, the ground-truth score may then be used to train an AI model of the AI module 180 of the monitoring system 100A to associate the ground-truth score to the overall disinfection quality or disinfection quality for each step of the disinfection process. For example, the seventh step 514 may calculate a satisfactory level of disinfection for each predetermined part/area of the user's fingers and/or one or both hands, which may make use of a percentage of the surface area of one or more predetermined parts/areas that have been cleaned from the previous step 512 or any other metric as the groundtruth score

The seventh step 514 may be advantageous not only regarding handwashing, regarding which there is substantial disagreement as to the proper steps, order of steps, and duration of steps, but also regarding other disinfection procedures regarding which there may not be a consensus regarding “best practices” or quantifiable observations regarding quality of procedures. This may include without limitation the disinfection of various pieces of equipment, such as medical or dental equipment or food preparation-related equipment, the disinfection of other parts of the human body, the disinfection of spaces such as a kitchen, operating room, or otherwise.

Embodiments of the present disclosure may comprise or utilize a special-purpose or general-purpose computer system that includes computer hardware, such as, for example, one or more processors and system memory, as discussed in greater detail below. Embodiments within the scope of the present disclosure also include physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, embodiments of the disclosure can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the disclosure.

Transmission media can include a network and/or data links which can be used to carry program code in the form of computer-executable instructions or data structures, and which can be accessed by a general-purpose or special-purpose computer system. A “network” may be defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RANI and/or to less volatile computer storage media at a computer system. Thus, it should be understood that computer storage media can be included in computer system components that also (or even primarily) utilize transmission media.

Computer-executable instructions may comprise, for example, instructions and data which, when executed by one or more processors, cause a general-purpose computer system, special-purpose computer system, or special-purpose processing device to perform a certain function or group of functions. Computer-executable instructions may be, for example, binaries, intermediate format instructions such as assembly language, or even source code.

The disclosure of the present application may be practiced in network computing environments with many types of computer system configurations, including, but not limited to, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

The disclosure of the present application may also be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

Some embodiments, such as a cloud-computing environment, may comprise a system that includes one or more hosts that are each capable of running one or more virtual machines. During operation, virtual machines emulate an operational computing system, supporting an operating system and perhaps one or more other applications as well. In some embodiments, each host includes a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources including processing capacity, memory, disk space, network bandwidth, media drives, and so forth.

By providing a disinfection system and method for using the same according to disclosed embodiments, the problem of existing attempts to monitor handwashing or other disinfection processes being poorly adapted to protect privacy and being ineffective and/or inefficient at monitoring the quality of handwashing is addressed. The disinfection system and method embodiments advantageously quantify the quality of handwashing and other disinfection events and simultaneously protect a user's privacy, facilitating the widespread adoption and roll-out of the system where otherwise such efforts to automate the monitoring of disinfection events in sensitive environments such as restrooms would be challenged. Managers are enabled by the disinfection system embodiments to ensure that employees, customers, or visitors are thoroughly completing disinfection procedures that may be required by local health ordinances or in order to keep the business, school, or establishment open during quarantine.

The disinfection system embodiments additionally enable robust detection of one or more poses of a disinfection procedure using one or both of an amplitude and a duration of the one or more poses, enabling improved granularity of detection. Additionally, by providing a disinfection system utilizing in embodiments human keypoint or key area detection in parallel with hand and/or finger detection, improving the reliability of detection by facilitating an estimate of a pose even when one or both hands is partially or entirely occluded or otherwise undetectable in one or more frames of a video feed.

Not necessarily all such objects or advantages may be achieved under any embodiment of the disclosure. Those skilled in the art will recognize that the disclosure may be embodied or carried out to achieve or optimize one advantage or group of advantages as taught without achieving other objects or advantages as taught or suggested.

The skilled artisan will recognize the interchangeability of various components from different embodiments described. Besides the variations described, other known equivalents for each feature can be mixed and matched by one of ordinary skill in this art to construct or use a disinfection system under principles of the present disclosure. Therefore, the embodiments described may be adapted to disinfection systems in general, including devices, systems, components, and methods for disinfecting hands or other body parts, objects, spaces, or otherwise.

Although the disinfection system has been disclosed in certain preferred embodiments and examples, it therefore will be understood by those skilled in the art that the present disclosure extends beyond the disclosed embodiments to other alternative embodiments and/or uses of the disinfection system and method for using the same and obvious modifications and equivalents. It is intended that the scope of the present disinfection system disclosed should not be limited by the disclosed embodiments described above, but should be determined only by a fair reading of the claims that follow.

1. A method for performing monitoring of a disinfection system, the method comprising:
receiving, at a controller, a captured image or video of a disinfection procedure from at least one image capture device; and
performing detection and detection-based tracking of a user's fingers and/or one or both hands from the captured image or video to determine a quality of the disinfection procedure.

2. The method of claim 1, wherein the detection and detection-based tracking of the user's fingers and/or one or both hands is performed using keypoint or key area based detection.

3. The method of claim 2, wherein the keypoint or key area based detection is used by an Artificial Intelligence (AI) module associated with the controller to determine a poses of the user's body, and/or fingers and/or one or both hands in each frame of the captured image or video while performing the disinfection procedure.

4. The method of claim 2, further comprising:
performing human keypoint or key area detection on a part of the user's body other than the user's fingers and/or one or both hands in addition to performing the keypoint or key area based detection on the user's fingers and/or one or both hands, wherein the human keypoint or key area detection is performed when one of the user's fingers and/or one or both hands are occluded.

5. The method of claim 1, further comprising:
receiving an identification credential from the user, the identification credential being configured to identify the user.

6. The method of claim 1, further comprising:
performing an operation to remove any identifying information of the user from the captured image or video such the identity of the user is hidden for a viewer of the captured image or video.

7. The method of claim 1, further comprising:
analyzing the captured image or video to obtain a set of metrics related to the performance of the disinfection procedure.

8. The method of claim 1, wherein detection alone, without the need for detection-based tracking, is used to determine the quality of the disinfection procedure.

9. A disinfection monitoring system, comprising:
at least one image capture device;
at least one processor;
a controller configured to receive a captured image or video from the at least one image capture device; and
at least one Artificial Intelligence (AI) module configured to perform detection and detection-based tracking of a user's fingers and/or one or both hands from the captured image or video to determine a quality of the disinfection procedure.

10. The disinfection monitoring system of claim 9, wherein the system further comprises one or more of:
an identification module configured to receive an identification credential from a user; and
a motion detection module configured to detect a presence of a user proximate the at least one image capture device.

11. The disinfection monitoring system of claim 9, wherein the controller is configured to identify, and blur identified human features of the user from the captured video or image.

12. The disinfection monitoring system of claim 9, wherein the controller is configured to provide a compliance message to the user through a user interface proximate the at least one image capture device.

13. The disinfection monitoring system of claim 9, wherein the controller is configured to provide animations or illustrations of each step of the disinfection process through a user interface proximate the at least one image capture device.

14. The disinfection monitoring system of claim 9, wherein the detection and detection-based tracking of the user's fingers and/or one or both hands is performed using keypoint or key area based detection.

15. The disinfection monitoring system of claim 14, wherein the keypoint or key area based detection is used by the AI module to determine a pose of the user's body and/or fingers and/or one or both hands in each frame of the captured image or video while performing the disinfection procedure.

16. The disinfection monitoring system of claim 14, wherein the AI module performs human keypoint or key area detection on a part of the user's body other than the user's fingers and/or one or both hands in addition to performing the keypoint or key area based detection on the user's fingers and/or one or both hands, wherein the human keypoint or key area detection is performed when one of the user's fingers and/or one or both hands are occluded.

17. A method for collecting data related to a disinfection procedure performed by a user at a disinfection system, the method comprising:
receiving, at a controller, a plurality of captured images or videos of a disinfection procedure from at least one image capture device;
generating a ground-truth score for each of the plurality of captured images or videos, the ground-truth score indicating a quality of the overall disinfection procedure and/or a quality of each step in the overall disinfection procedure; and
using the ground-truth score for each of the plurality of captured images or videos as training data to train one or more Artificial Intelligence (AI) modules to perform monitoring of the disinfection procedure.

18. The method of claim 18, wherein the ground-truth score is generated by one of a human evaluator or by a disinfection data collection system.

19. The method of claim 18, wherein the ground-truth score is obtained using a UV light system of a disinfection data collection system by performing the following steps:
providing a disinfection system proximate a disinfection area;
dispensing a predetermined quantity of a fluorescent or UV-active material to at least one target surface;
capturing a first set of images of the at least one target surface under a light source prior to initiating a disinfection procedure;
capturing at least one video of the disinfection procedure;
capturing a second set of images of the target surface under the light source after the disinfection procedure; and
determining a percentage of an area of the at least one target surface that is cleaned of the fluorescent or UV-active material.

20. The method of claim 19, further comprising the step of determining a procedure for satisfying a disinfection requirement, wherein the disinfection requirement is a predetermined percentage of the area of the at least one target surface that is cleaned of the fluorescent or UV-active material.
